(19) United States
(12) Patent Application Publication
(10) Pub. No.: US 2005/0071001 A1
(43) Pub. Date: Mar. 31, 2005

(54) Artificial heart power and control system

(76) Inventor: Robert Jarvik, New York, NY (US)

Correspondence Address: Mark P Levy, ESQ;Thompson Hine LLP, 2000 Courthouse Plaza NE, 10 W. Second Street, Dayton, OH, 45402-1758, US

(21) Appl. No.: 10/674,861

(22) Filed: Sep. 30, 2003

(51) Int. Cl. A61M 1/12

(52) U.S. Cl. 623/3.28; 623/3.27; 600/17

(57) ABSTRACT

The present invention provides a human engineered power and control system for artificial hearts or assist devices configured for ease of use, ruggedness, and high reliability. Battery powered systems of the prior art have required multiple cables and connectors that are subject to failure due to damage or wear. In the present invention, direct connection of the batteries to the control system eliminates multiple cables and connectors used with previous designs. A novel method of connecting batteries to the control system and exchanging batteries without interruption of power is provided in a compact user friendly configuration. The control system may provide periodic reductions in assist device flow to permit the natural ventricle to eject blood through the natural outflow valve, open the valve leaflets to prevent them from adhering together, and achieve sufficient washout to prevent thrombosis. Using either software based control or software independent electronic circuitry, the flow pumped by the artificial heart is reduced for a long enough period of time to permit at least a few beats of the natural heart to generate sufficient pressure to open the outflow valve. In a control system embodiment in which the patient manually adjusts the pump speed to incremental settings for rest and exercise conditions, a pulsatile flow mode is disclosed which provides approximately the same flow at a given incremental setting as the pump produces when running in a constant speed mode at the same setting. As the patient learns which speed setting is best for daily activities, the patient may use the same setting with either a pulsatile or constant pump speed mode.

### BACKGROUND OF THE INVENTION

Artificial hearts which support the right or left ventricles are blood pumping devices connected so that they receive blood from the atrium or ventricle, and pump it into the aorta (for a left heart assist device) or the pulmonary artery (for a right heart assist device). Electric rotary artificial hearts use axial flow pumps, centrifugal or mixed flow pumps powered by batteries to provide mobility and rehabilitate patients to a high quality of life. As patients resume a near normal life, the human engineering of the equipment they wear becomes an important aspect of their safety and comfort. External power and control systems must meet the physiologic needs of the patient to adjust the blood flow according to the level of exercise of the patient and other factors. Either the flow must be adjusted automatically using a computational algorithm, or it must be adjusted manually.

If the device captures all of the flow entering the natural ventricle, and therefore all of the flow passes through the device (for the side of the heart to which the device is applied), then the natural heart's outflow valve will not open. This can cause several serious problems, including blood clots near the valve leaflets, which may break free to cause thromboemboli, or may become infected. If the device captures all the flow for a prolonged period of time, even if no thrombi form, the natural valve leaflets may fuse together, complicating removal of the device if the natural heart recovers, or hindering the natural heart's ability to serve the function of emergency life support if the artificial heart malfunctions.

The present invention provides human engineered power and control systems configured for ease of use, ruggedness, and high reliability. Direct connection of the batteries to the control system eliminates cables and connectors used with previous designs, which are prone to failure, particularly where the cable joins the connector. Periodic reductions in assist device flow is provided to permit the natural ventricle to eject blood through the natural outflow valve, open the valve leaflets to prevent them from adhering together, and achieve sufficient washout to prevent thrombosis. Using either software based control or software independent electronic circuitry, the flow pumped by the artificial heart is reduced or stopped completely for a long enough period of time to permit at least a few beats of the heart to generate sufficient pressure to open the outflow valve. Depending on the type of artificial heart used and the hemodynamic conditions, the period of time of reduced device flow may vary from a few seconds, up to thirty seconds or more. This differs from providing pulsatility to an assist device at a frequency within a wide physiologic range (˜40->200 BPM), which mimics natural conditions and can be sustained indefinitely without the natural outflow valve opening.

When used with a rotary type of blood pump, the present invention also provides a mechanism to avoid prolonged blockage of the inflow due to suction produced by the pump. For example, if an axial or centrifugal blood pump uses an inlet cannula placed within the left ventricle, and if the venous return of blood to the left ventricle is less than the device would pump at the speed and differential pressure conditions existing at a particular moment, then the pump will generally produce a negative pressure at the inlet. This can cause the natural tissues of the heart, such as the ventricular wall, or mitral valve leaflets to occlude the opening to the device, which will markedly increase the suction at the cannula opening and firmly hold the obstructing tissue in place. Unless the pump speed is reduced enough to sufficiently reduce the suction pressure, the blockage will continue. The natural heart could continue to beat, and could eject through the aortic valve, while the pump could be blocked and could thrombose. Intermittently reducing the pump speed low enough to permit back flow from the aorta, will release an obstruction caused by suction.

For decades there have been numerous articles in the scientific literature addressing the issue of pulsatility with rotary blood pumps. Some prior art devices have varied the pump speed to generate a pulsatile flow. Some data suggests that pulsatile flow is necessary to prevent abnormal physiology including accumulation of tissue edema, fluid volume accumulation, and hypertension. Other studies indicate that long term pulseless flow is well tolerated and fluid shifts seen with short term non-pulsatile flow become corrected to near normal after one to two months. Most systems developed to produce pulsatility for rotary blood pumps have attempted to mimic near normal aortic pressure waveforms so that the pressure sensed by the baroreceptors will be as close to normal as possible. The wave forms of pressure pulses of the present invention are not intended to mimic the normal aortic waveform, but rather are to provide conditions which help the weak natural heart open the aortic valve to prevent thrombus or to provide pulsatility in the aortic root to increase washing and prevent thrombus, and also to increase wall motion of the ventricle to decrease the thrombus risk within the ventricle.

The preferred mode of varying pump speed provided by the present invention, is an intermittent low speed, low flow mode, where the ventricular unloading effect of the assist pump is so diminished that the natural heart can sufficiently fill to be able to eject blood across the aortic valve into the aortic root. The waveform produced by the pump slowdown and then the pump speedup after several natural heartbeats is nothing like a normal aortic waveform, and actually distorts the waveforms of two natural heartbeats.

This provides variation in the flow around the aortic valve which helps prevent stagnant conditions which pre-dispose to thrombus. With a manually adjusted speed control system, which uses a dial to set the pump speed in fixed increments, it is helpful for the overall flow at a given speed setting to be approximately the same if either a pulsatile mode or a non-pulsatile mode is used. The present invention accomplishes this, which makes it easy for the patient to learn which speed setting to use for different levels of exercise, without having to pay attention to whether the controller is using the pulsatile mode or the non-pulsatile mode.

### OBJECTS OF THE INVENTION

1. It is an object of the present invention to provide an electronic control system which will periodically reduce the flow produced by a rotary blood pump implanted between the left ventricle and the aorta, to a low enough level that the natural heart will eject blood through the aortic valve, and will do this frequently enough to provide sufficient washout to reduce the risk of thrombus formation around the valve and the aortic root.
2. It is a further object of the invention to provide a left heart assist device control system which will decrease the incidence of blood clot formation around the aortic valve by providing pulsatile flow.
3. Another object of the invention is to prevent obstruction of the inflow for more than a brief period of time due to suction of tissue over the inflow opening to the pump or due to suction of the atrial wall over the mitral annulus.
4. It is a further object of the invention to provide an artificial heart power and control system to which two battery packs may be simultaneously connected without the use of cables to improve the reliability of the system.
5. It is a still further object of the invention to provide an artificial heart power and control system which permits batteries to be changed without stopping the device and requires no cables connecting the batteries to the controller.
6. It is a still further object of the invention to provide all of the above described characteristics in a highly compact rugged design comfortably worn by the patient.
7. It is another object of the invention to provide connectors for connecting batteries to an artificial heart control system, which retain contact when rotated.
8. It is a further object of the present invention to provide a rotational locking mechanism to securely attach the batteries to the control system.

### THE FIGURES

FIG. 1 is a simplified line drawing illustrating an intraventricular left heart assist device connected to one embodiment of a control and power system of the present invention. The control and power system is shown enlarged compared to the illustration of the patient.

FIG. 2 is a block diagram of the power and control system showing two connectors and two batteries.

FIGS. 3A, 3B, & 3C are three dimensional illustrations of the controller and one battery showing how they are connected.

FIGS. 3A top, 3B top, and 3C top are top view illustrations of the controller and batteries corresponding to FIGS. 3A-3C.

FIG. 4A is a section of a co-axial connector and rotationally actuated locking mechanism, showing the male connector co-axial plug affixed to the battery case and the co-axial receptacle in the controller case. The battery case and controller cases are shown in a rotational position with relation to each other similar to the position shown in FIG. 3A.

FIG. 4B is a section of a co-axial connector and rotationally actuated locking mechanism, showing the battery case and controller case attached together in position very close to that illustrated in FIG. 3C. The controller case is shown in the same position in both FIGS. 4A and 4B. The battery case is rotated approximately 45 degrees in FIG. 4B compared to FIG. 4A.

FIGS. 5A-5H is a series of top view drawings of the outline of the controller and batteries during the sequential steps of changing batteries.

FIGS. 6A & 6B are graphs of the motor and pump speed changes over time used with the intermittent pump flow reduction function of the present invention.

FIGS. 7A & 7B are graphs of motor (pump) speed changes used with the pulsatile flow function of the present invention.

### SPECIFIC DESCRIPTION OF THE INVENTION

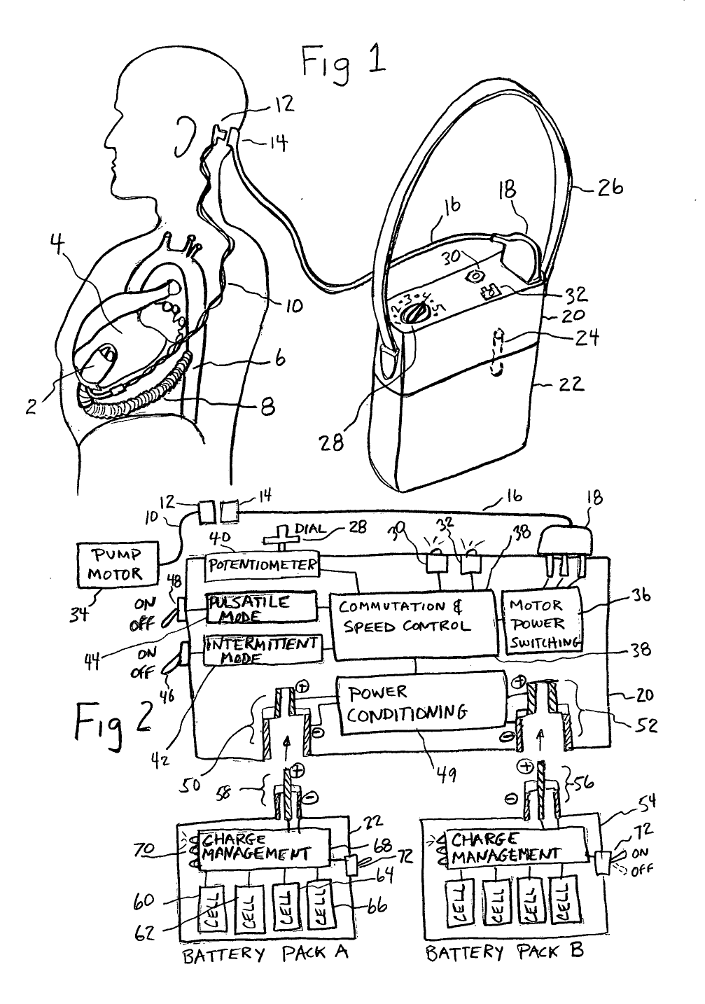

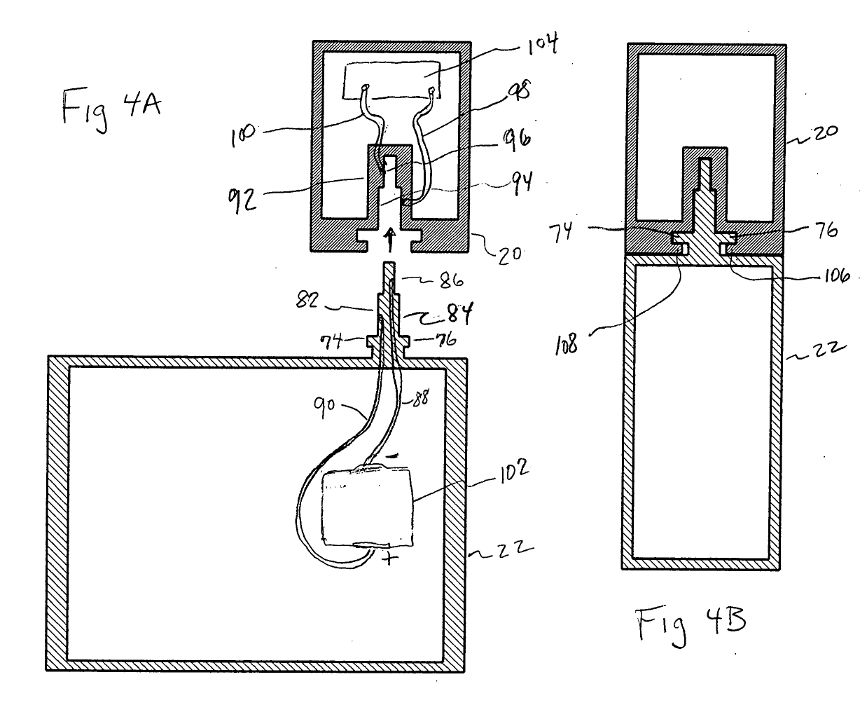

FIG. 1 illustrates the preferred embodiment of the invention. A rotary left heart assist axial flow blood pump 2 is implanted into the left ventricle 4 and connected to the aorta 6 by a vascular graft 8. A three phase electric motor which is contained within the blood pump 2 is powered via three wires of an implanted power cable 10. The cable crosses the skin at a percutaneous lead 12 which may include a connector 14 by which it is attached to an external power cable 16 having a plug 18 which permits the individual wires of the cable to be connected to the electronics control system 20. A battery pack which may contain multiple cells and will be referred to in this disclosure as simply a battery, is removably connected to the control system 20 by a coaxial connector 24, which is comprised of a receptacle—(FIG. 4) mounted directly within the control system and a plug—(FIG. 4) mounted within the battery. When mounted together as shown, the control system and battery form an integral unit configured to be comfortably worn like a purse or camera case by means of a shoulder strap 26. A manually adjustable speed control dial 28 may be located on the upper side of the control system as well as alarm and other indicator lights 30, 32.

Since the control system and battery connect directly together, only one cable is necessary to connect this integral unit to the implanted components of the system. In previous systems patients would require a total of three or four external cables and also needed to keep additional backup cables with them at all times. This became cumbersome to deal with. Cables could become tangled and it was difficult to keep them neat to prevent damage.

The compact configuration of this embodiment is also preferred because the control system case and battery case can be made strong and impact resistant. With only one cable, if the system is dropped, it is less likely that it will damage a cable connector than other systems which use three cable connectors. The coaxial connector, by means of which the battery and control system are electrically connected, is located between their cases and is thus protected from direct impact.

FIG. 2 is a block diagram of the controller and battery pack circuitry, connectors, and indicators. The brushless dc motor 34, which powers the blood pump, is connected to the controller 20, by connectors and cables 18, 16, 14, 12, and 10, described above. In the preferred embodiment a three phase motor is used, and within cable 16, a separately insulated wire is included for each phase of the motor. The three pin plug 18, connects the motor wires to the switching circuitry 36 which includes power transistors to switch the power to each motor phase on and off according to the proper timing as determined by the commutator and control portion of the circuitry 38. It is preferable to utilize analog circuitry with no microprocessor. This avoids the need for any software, and eliminates any potential software related failure modes. If an embodiment is used which does have a microprocessor and does require software, thorough software validation is required. A speed control dial 28 is mounted to the controller case and may have spring loaded ball and detent set positioning so that a fixed number of incremental rotational positions can be set by the dial. As the dial is rotated ten degrees, for example, the spring loaded ball engages a detent and holds the dial in that position unless turned to another detent position. The dial is fixed to the shaft of a single turn potentiometer 40, which gives a different resistance value for each detent position. The potentiometer is connected to the commutator circuitry 38, which runs the motor at a predetermined speed in proportion to dial set resistance. In other embodiments, other speed setting methods may be used, such as a multi-position switch together with a group of fixed value resistors, or digital signals derived from a microprocessor based digital system with a visual display.

The controller also includes circuits to product the intermittent low speed mode 42 and pulsatile mode 44. These modes are each engaged or disengaged by manually throwing switches 46 and 48 to either the on or off position, or by jumper connections. These functions will be further discussed below.

The controller also includes power conditioning circuitry 49, into which the battery pack output is connected by means of two coaxial receptacles, 50, 52. The power conditioning circuitry includes a transformer to produce the necessary regulated voltage to run the commutator chip and other circuitry and connections to bring full voltage power directly from the batteries to the power transistors. It also includes schottky diodes to prevent discharge of one battery pack into the other pack when two packs are connected simultaneously, as illustrated when both battery pack A, 22, and battery pack B, 54, are plugged in by means of co-axial plugs 56, and 58.

Each battery pack must provide sufficient voltage for the motor, which in the preferred embodiment requires approximately 10-16 volts. Four Li-ion cells, 60,62,64,66, may be used per pack, as illustrated. If Li-ion cells are used, it is advantageous to utilize a battery power management circuit, 68. This may include LED indicator lights, 70, which show the approximate amount of power remaining in the pack when a switch, 72, is closed.

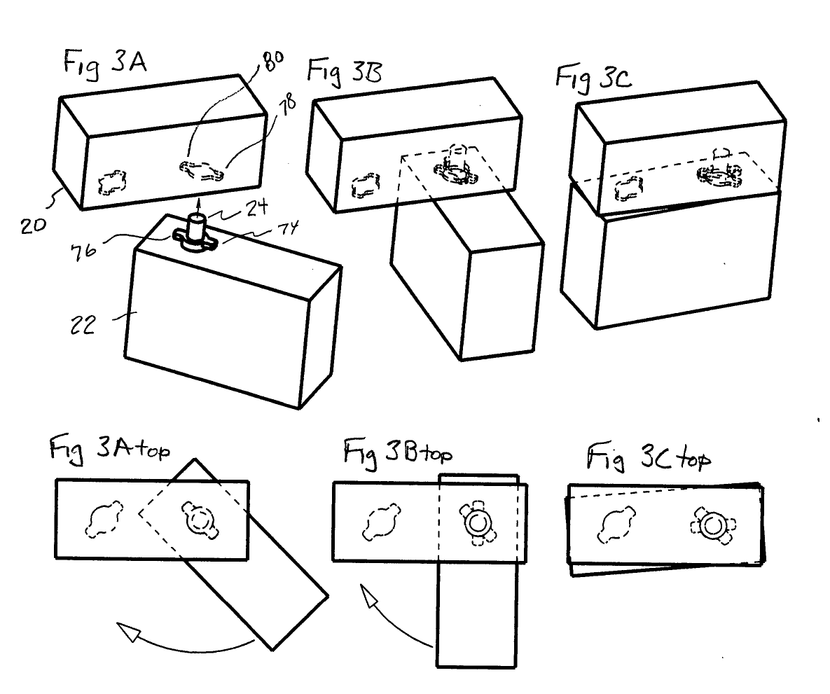

FIGS. 3A, 3B, 3C and FIGS. 3A top, 3B top, and 3C top, illustrate the attachment of a battery pack to the controller. The battery pack includes a protruding co-axial plug 24 and rotational locking extensions 74,76. The locking extensions fit into slots 78, 80 on both sides of the co-axial receptacle on the controller. When the battery 22 is separate from the controller 20 as shown in FIG. 3A, and is rotated to the correct angle with relation to the controller, the co-axial plug can be inserted all the way into the co-axial receptacle and the locking extensions will pass through the locking mechanism slots. Then if the battery pack is rotated from the position shown in FIG. 3B to the position shown in FIG. 3C, the battery pack will be attached onto the controller. It can then only be removed by rotating it.

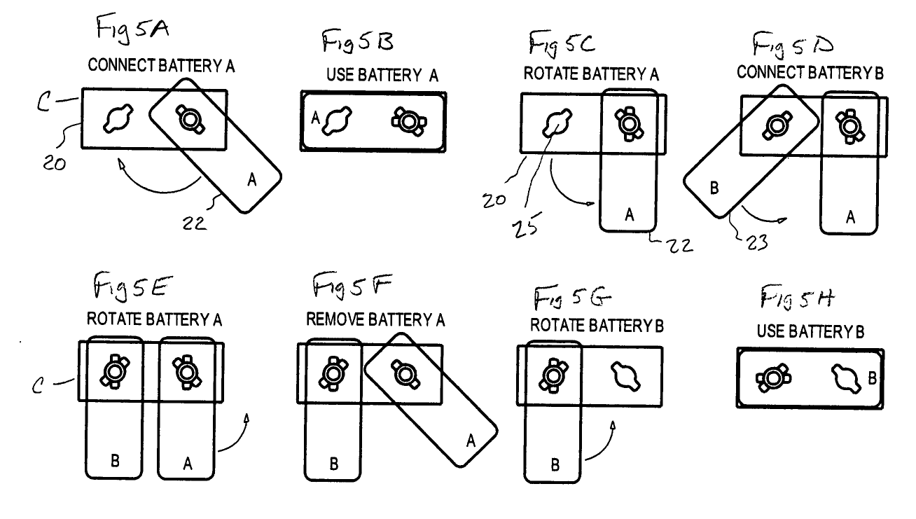

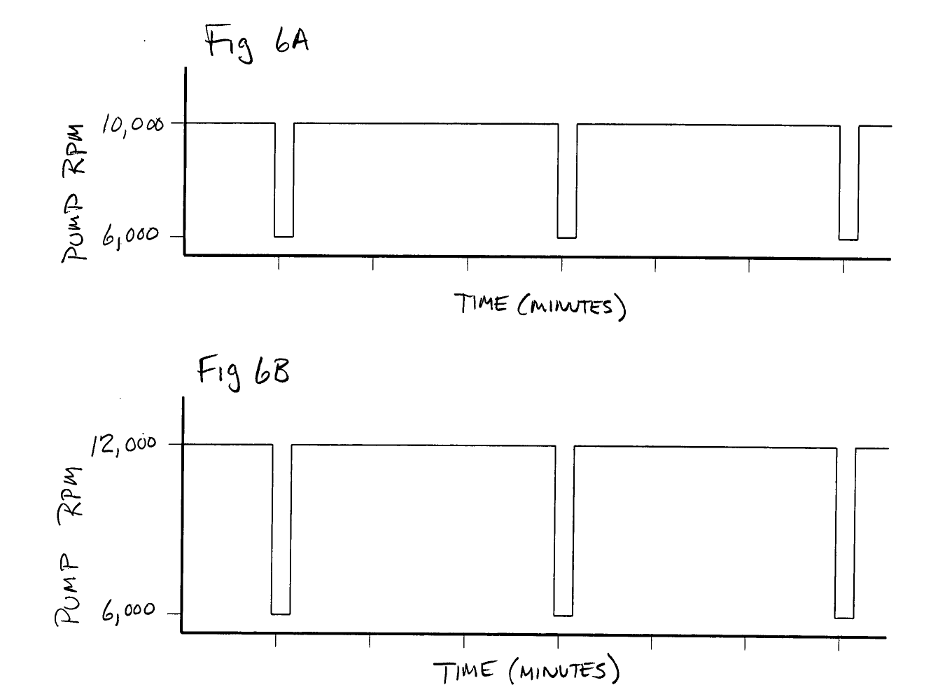

FIG. 4A is a section of a co-axial connector and rotationally actuated locking mechanism, showing the male connector co-axial plug affixed to the battery case and the co-axial receptacle in the controller case. The battery case and controller cases are shown in a rotational position with relation to each other similar to the position shown in FIG. 3A. The co-axial connector has a plug, 82, which is shown with a larger diameter contact portion, 84, and a smaller diameter contact portion, 86. These portions are insulated from one another and connected to wires, 88,90, which are in turn connected to the battery positive and negative contacts. The receptacle portion of the connector, 92, has contact surfaces, 94,96, which are insulated from one another and mate with the plug, 82, to provide electrical connection when the connector parts are mated. Wires 98, 100, provide electrical connection from the connector to the circuit board 104. The plug of the connector is mounted to a base having two locking extensions, 74,76, which fit into slots 78,80, (best seen in FIG. 3 and FIG. 5) when the battery is rotated to the proper position relative to the controller. This rotational position is represented in FIG. 4A, and so the plug and receptacle can be mated by pushing the plug into the receptacle as illustrated by the arrow in FIG. 4A. When this is done, the connector makes contact and power is supplied from the battery, 102, to the controller board, 104. This connection is maintained when the battery pack case, 22, together with the locking extensions, 74,76, are rotated relative to the controller case, 20. The battery pack case and controller case are then retained together by the rotational capture of the locking extensions 74,76, by portions of the controller case, 106,108, with which they engage. Thus, the structure of the co-axial connector together with the rotationally engaged locking mechanism provides the ability to maintain electrical connection of the connector in different rotational positions of the controller case relative to the battery pack case. This permits the series of positions necessary to change batteries without disconnecting power from the controller.

FIGS. 5A-5H diagram the sequence of positions used in the process of changing batteries. The controller is designated C and the two battery packs are designated A and B. In FIG. 5A battery A is plugged into the controller and rotated to its in use position shown in FIG. 5B. This position is like the controller battery combination shown in FIG. 1. When the user is ready to change battery pack A for battery pack B, pack A is first rotated to the position shown in FIG. 5C. Note that in this position, the locking mechanism still retains pack A, 22 together with controller 20. Battery pack B, 23, is then attached to the controller by inserting its co-axial plug into the open receptacle, 25, on the controller, 20, as shown in FIG. 5D, and rotating battery pack B, 23, 45 degrees to the position shown in FIG. 5E. Both battery packs are now locked onto the controller. Next, pack A is rotated 45 degrees to the position shown in FIG. 5F and then removed by pulling the plug out of the receptacle. This leaves only battery pack B attached to the controller shown in FIG. 5G. Then, battery pack B is rotated to its final use position as shown in FIG. 5H. If the user wishes, the controller may be left attached to both battery packs as shown in FIG. 5E for a long period of time, such as overnight.

FIG. 6 illustrates the intermittent low speed mode which helps prevent thrombus formation. FIG. 6a shows a typical motor speed cycle with the pump speed setting dial adjusted to 10,000 RPM. Once every three minutes, the speed is reduced to 6,000 RPM for approximately 15 seconds, and then is returned to 10,000 RPM. During the time the speed is reduced, the natural heart will typically beat five to ten times. Since the pump will not be able to capture the full flow of the natural heart during this time, because the flow the pump can produce at this reduced speed is not high enough, the natural heart will eject blood through the aortic valve in most patients. This will help provide sufficient washing of the aortic valve to avoid thrombus when combined with adequate anticoagulation.

FIG. 6b, shows that when the speed of the blood pump is higher than the 10,000 RPM value illustrated in FIG. 6a, during the period of slowdown, the speed will still be lowered to 6,000 RPM. Therefore, during the slow down time, the patient's natural heart will behave approximately the same no matter what speed the pump had been set to prior to the brief slowdown period.

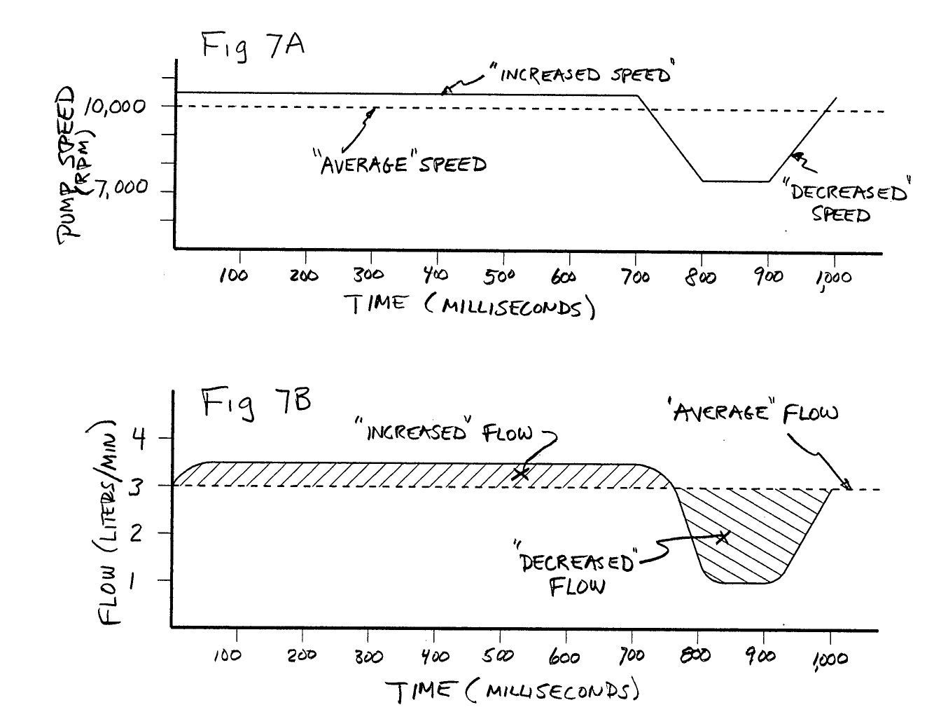

FIG. 7 shows a representative speed cycle for the pulsatile function of the controller. In this example, the frequency of the speed variations created by the controller is 60 per minute. The time per cycle is one second, which is 1000 milliseconds. The heavy solid line represents the speed waveform which is selected so that the flow produced by the pump under the varying speeds of the cycle will be approximately the same as it would have been had the speed been held constant at the speed dial setting used. In this example the speed dial setting is at 10,000 RPM, as illustrated by the dotted line in the FIG. 7a. The solid line illustrates the speed variation over the cycle for the pulsatile control mode. For the first 700 msec. the pump is run at 10,500 RPM and pumps somewhat more than it would at 10,000 RPM. Between 700 msec. and 950 msec. the pump speed is decelerated down to 7,000 RPM. and the flow drops markedly. The speed is then rapidly re-accelerated to 10,500 and the flow increases rapidly. As shown by the crosshatched area, for about the first 750 msec. the flow is higher than it would have been at a constant speed of 10,000 RPM, and for the rest of the cycle it is lower. If the waveform of the pump speed is of the general nature shown, and if the time and amplitude of the speed changes over the cycle are selected appropriately for the specific pump design used, then a pulsatile flow cycle is obtained which produces approximately the same average flow as the pump would produce if it ran at a constant speed. This is the preferred type of pulsatile waveform for the pulsatile flow control mode of the present invention.

The control circuit may also vary the amplitude of the high and low values reached over the pulsatile cycle as a percentage of the speed set by the dial, so that the mean flow during either the constant pump speed mode or the pulsatile pump speed mode approximately match for each of the speed settings available on the dial.

The same principal of increase in pump speed during a high speed period of time (compared to the speed at a particular dial setting for a constant speed mode) may be used with any pulsatile frequency, such as the intermittent low speed mode illustrated in FIG. 6, to achieve approximately the same flow for the same dial setting in either the constant speed or variable speed mode. The control system is also provided with circuitry and a switch which allows it to be run in either the pulsatile or intermittent low speed mode, or in a constant speed mode. The circuitry includes calibration potentiometers or other calibration devices which permit the system to be calibrated so that the flow obtained at a particular dial setting is approximately the same regardless of which mode of the controller is used.

The information disclosed in the description of the present invention is intended to be representative of the principles I have described. It will thus be seen that the objects of the invention set forth above and those made apparent from the preceding description are efficiently obtained and that certain changes may be made in the above articles and constructions without departing from the scope of the invention. It is intended that all matter contained in the above description and shown in the accompanying drawings shall be interpreted as illustrative but not in a limiting sense. It is also understood that the following claims are intended to cover all of the generic and specific features of the invention herein described and all statements of the scope of the invention which, as a matter of language, might be said to fall there between.

1. An element ranking method, comprising:
aggregating business related data using a common data dictionary,
identifying one or more business value indicators for one or more elements of value using at least a portion of the data, and
determining element of value rankings by calculating the combined relative strength of the indicators for each element of value.

2. The method of claim 1 that further comprises, using the element of value rankings to support the valuation of elements of value.

3. The method of claim 1 where the elements of value are brands, customers, employees, production equipment, strategic partnerships, vendors, vendor relationships and combinations thereof.

4. The method of claim 1 wherein the business value indicators are selected from the group consisting of element data trends, element data ratios, element data summaries, element data rates of change, element data patterns, element data averages, element data, time lagged element data trends, time lagged element data ratios, time lagged element data summaries, time lagged element data rates of change, time lagged element data patterns, time lagged element data averages, time lagged element data, composite variables and combinations thereof.

5. The method of claim 1 where business related data is obtained from advanced financial systems, basic financial systems, operation management systems, sales management systems, human resource systems, accounts receivable systems, accounts payable systems, capital asset systems, inventory systems, invoicing systems, payroll systems, purchasing systems, the Internet, user input, external databases and combinations thereof.

6. The method of claim 1 where the business related data is obtained for two or more businesses in the same industry.

7. The method of claim 1 where the data dictionary defines standard data attributes from the group consisting of account numbers, components of value, currencies, elements of value, enterprise designations, time periods, units of measure and combinations thereof.

8. The method of claim 1 where data envelopment analysis (DEA) analysis is used to identify the relative strength of the indicators for each element of value.

9. A computer readable medium having sequences of instructions stored therein, which when executed cause the processors in a plurality of computers that have been connected via a network to perform an element management method, comprising:
aggregating business related data using a common data dictionary,
identifying one or more tangible business value indicators for one or more elements of value using at least a portion of the data,
calculating concrete measures of element impact on aspects of financial performance using said indicators,
creating an enterprise financial performance model using said measures, and
determining and the list of changes to element business value indicators that will maximize value using said model.

10. The computer readable medium of claim 9 where the method further comprises the net contribution of each element of value to a value of the enterprise using said model.

11. The computer readable medium of claim 9 where the elements of value are brands, customers, employees, production equipment, strategic partnerships, vendors, vendor relationships and combinations thereof.

12. The computer readable medium of claim 9 where the brands, partnerships and vendor relationships further comprise enterprise intellectual capital.

13. The computer readable medium of claim 9 wherein the business value indicators are selected from the group consisting of element data trends, element data ratios, element data summaries, element data rates of change, element data patterns, element data averages, element data, time lagged element data trends, time lagged element data ratios, time lagged element data summaries, time lagged element data rates of change, time lagged element data patterns, time lagged element data averages, time lagged element data, composite variables and combinations thereof.

14. The computer readable medium of claim 9 where business related data is obtained from advanced financial systems, basic financial systems, operation management systems, sales management systems, human resource systems, accounts receivable systems, accounts payable systems, capital asset systems, inventory systems, invoicing systems, payroll systems, purchasing systems, the Internet, user input, external databases and combinations thereof.

15. The computer readable medium of claim 9 where the measures are selected from the group consisting of value drivers, composite variables, vectors, relative element strengths, element rankings and combinations thereof.

16. The computer readable medium of claim 15 where data envelopment analysis (DEA) analysis is used to identify the relative strength of the indicators for each element of value.

17. The computer readable medium of claim 9 where the business related data is obtained for two or more businesses in the same industry.

18. The computer readable medium of claim 9 where the data dictionary defines standard data attributes from the group consisting of account numbers, components of value, currencies, elements of value, enterprise designations, time periods, units of measure and combinations thereof.

19. The computer readable medium of claim 9 where the aspects of financial performance are selected from the group consisting of revenue, expense, capital change, current operation value, real option value, market sentiment value, total value and combinations thereof.

20. The computer readable medium of claim 9 where identifying one or more business value indicators for one or more elements of value further comprises
creating one or more tangible indicators of element impact on aspects of enterprise financial performance and
using a series of models to identify causal indicators of element impact on one or more aspects of enterprise financial performance.

21. A computer readable medium having sequences of instructions stored therein, which when executed cause the processors in a plurality of computers that have been connected via a network to perform a performance measure method, comprising:
aggregating enterprise related data,
identifying tangible indicators of element impact on enterprise intellectual capital,
developing solid measures of element impact on enterprise intellectual capital using one or more of said indicators, and
producing enterprise performance management information using at least one of the measures.

22. The computer readable medium of claim 21 where the method further comprises making the enterprise performance management information available for review and use via a paper document or electronic display.

23. The computer readable medium of claim 21 where an enterprise is a single product, a group of products a division or a company.

24. The computer readable medium of claim 21 where data is aggregated using a common schema.

25. The computer readable medium of claim 21 where the performance management information further comprises an intellectual capital valuation.
